(19) United States
(12) Patent Application Publication
(10) Pub. No.: US 2024/0358300 A1
(43) Pub. Date: Oct. 31, 2024

(54) System for Line Draw Flushing and Blood Collection and Method of Use Thereof

(71) Applicant: Becton, Dickinson and Company, Franklin Lakes, NJ (US)

(72) Inventors: Jonathan Karl Burkholz, Salt Lake City, UT (US); Praveen Nalawade, Bangaluru (IN); Richard Bradley Timmers, Westwood, NJ (US)

(21) Appl. No.: 18/644,799

(22) Filed: Apr. 24, 2024

Related U.S. Application Data
(60) Provisional application No. 63/461,671, filed on Apr. 25, 2023.

(51) Int. Cl. A61B 5/15; A61M 39/00; A61M 39/02; A61M 39/10; A61M 39/18

(52) U.S. Cl. CPC A61B 5/150992; A61M 39/0208; A61M 39/18; A61M 2039/0018; A61M 2039/1033

(57) ABSTRACT

Provided herein is a system including a pre-filled syringe with a barrel defining a chamber containing a solution therein and a fluid connector component at the distal end of the barrel. A plunger assembly including a plunger and a stopper is movable within the barrel, with the plunger including a channel extending along a length thereof. An access device is positioned at the proximal end of the plunger and includes a receiving cavity and a fluid access component, the receiving cavity having an opening aligned with the channel and the fluid access component positioned over the opening and extending into the receiving cavity. A cannula is positioned within the barrel and extends through the stopper, the cannula placing the fluid connector component in fluid communication with the channel. A closure assembly is secured to the fluid connector component and includes a seal member configured to seal off a lumen thereof.

### CROSS-REFERENCE TO RELATED APPLICATION

The present application claims priority to U.S. Provisional Application No. 63/461,671, entitled “System for Line Draw Flushing and Blood Collection and Method of Use Thereof” filed Apr. 25, 2023, the entire disclosure of which is hereby incorporated by reference.

### BACKGROUND OF THE INVENTION

### Field of the Invention

The present disclosure relates to a system for line draw flushing and blood collection, along with an associated method of use thereof.

### Description of Related Art

Catheters are commonly used to administer fluids into and draw fluids (i.e., blood) out of the body, with patients in a variety of settings, including in hospitals and in home care, being administered such fluids or having blood drawn via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In some applications, a VAD is used for collection of a blood specimen in connection with performing of a blood culture. Blood cultures are often used as a tool to detect the presence of bacteria or fungi in a blood sample of a patient, to identify the type of bacteria or fungi present, and to direct the treatment of the patient. The current practice of collecting a venous blood sample for blood culture testing involves a venipuncture with a blood collection set with an attached luer-lock access device (LLAD). During the insertion process, the venipuncture device may pick up microbes from the skin and insertion process and later transfer such microbes into the blood culture sample leading to a false positive test result for sepsis. To minimize the risk of a false positive, a discard sample is typically taken to remove the initial volume of blood that more likely contains microbes from the skin, such as discarding the first 1-10 ml of blood drawn.

In obtaining a blood specimen such as when performing of a blood culture, a workflow process is implemented that is meant to sterilize connector components of the VAD and provide for collection of a venous blood sample. The workflow typically beings with a clinical practitioner disinfecting the access port (or a needle-free connector thereon) of the VAD, such as via a rubbing of the port with an alcohol wipe for example. Upon disinfecting of the access port, a pre-filled syringe (with a luer connector thereon) is connected to the port to flush the indwelling VAD, with the syringe then being disconnected from the VAD access port and discarded after flushing is complete. Upon disconnection of the syringe, the clinical practitioner again disinfects the access port/needle-free connector, such as via use of an alcohol wipe as previously described. Upon disinfecting of the access port, a LLAD is connected to the port via which a discard volume of blood is first drawn (via a diversion syringe or connection of a vacutainer tube to the LLAD, for example), before a blood culture sample is then drawn (such as via connection of another vacutainer tube to the LLAD, for example).

While the above-described workflow process provide for effective collection of a blood specimen (such as in connection with performing of a blood culture), the workflow process is labor intensive and increases the risk of introducing microbes to the patient and/or the blood collection sample due to the large number of steps and connections/disconnections performed in the workflow process. As one example, it is possible in some instances for the clinician to forget to wipe down the access port of the VAD prior to connection of the LLAD thereto and, if the disinfection of the access port is not performed properly, a false positive blood culture sample may arise.

Accordingly, a need exists in the art for a system and method of used thereof that provides a simplified workflow process for VAD flushing and line draw blood sample collection.

### SUMMARY OF THE INVENTION

Provided herein is a system for line draw flushing and blood collection. The system includes a syringe assembly having a pre-filled syringe including a barrel having a distal end and a proximal end and that defines a chamber containing a solution therein, and a fluid connector component at the distal end of the barrel. The syringe assembly also includes a plunger assembly including a plunger having a proximal end and a distal end, and a stopper positioned at the distal end of the plunger that is insertable into the proximal end of the barrel and slidable therein, the plunger including a channel extending along a length thereof between the proximal end and the distal end. The syringe assembly also includes an access device positioned at the proximal end of the plunger, the access device comprising a receiving cavity and a fluid access component, the receiving cavity having an opening therein aligned with the channel, and the fluid access component positioned over the opening and extending into the receiving cavity, the fluid access component in fluid communication with the channel. The syringe assembly also includes a cannula positioned at least partially within the barrel and extending through the stopper, with the cannula configured to place the fluid connector component in fluid communication with the channel. The syringe assembly also includes a closure assembly secured to the fluid connector component, the closure assembly comprising a seal member configured to seal off a lumen of the fluid connector component to contain the solution within the chamber of the syringe.

In some embodiments, the closure assembly comprises an end cap configured to engage the fluid connector component, wherein the fluid path seal is positioned within end cap, and wherein a proximal end of the end cap comprises a luer fitting configured to mate with an associated luer fitting of the fluid connector component.

In some embodiments, the closure assembly further includes a scrubbing cap secured to a distal end of the end cap, with the scrubbing cap comprising a housing secured to the end cap and defining a cavity, a scrubbing insert positioned within the cavity, and a seal attached over the cavity to seal the scrubbing insert within the cavity.

In some embodiments, the scrubbing insert comprises a resilient material including an antimicrobial solution or agent absorbed therein configured to disinfect a surface of the access port of the vascular access device.

In some embodiments, the housing comprises a connector portion including a threaded connection configured to engage with the end cap via a twist-type engagement.

In some embodiments, the closure assembly comprises a peel-off web coupled directly to the fluid path seal, to aid in removal of the fluid path seal from the fluid connector component.

In some embodiments, the system further includes a container defining a reservoir, the container positionable within the receiving cavity and engageable with the fluid access component to transfer fluid into the reservoir.

In some embodiments, the fluid access component comprises a needle defining a lumen therein, and wherein the container comprises a cover pierceable by the needle to place the reservoir in fluid communication with the channel of the plunger.

In some embodiments, wherein each of the receiving cavity and the proximal end of the plunger includes a luer connection, the luer connection of the receiving cavity engaging the luer connection of the plunger to secure the access device to the plunger assembly, and wherein a collapsible sleeve extending through the luer connections places the channel in fluid communication with the fluid access component.

In some embodiments, the plunger and barrel comprise a locking feature positioned and configured to lock the plunger relative to the barrel when the plunger assembly is in a distally advanced position.

In some embodiments, the plunger and barrel comprise a locking feature positioned to lock the plunger relative to the barrel when the plunger assembly is in a proximally retracted position.

In some embodiments, the cannula is coupled to the barrel and remains stationary during use of the syringe assembly.

In some embodiments, the cannula is coupled to the plunger, such that the cannula is movable responsive to advancing or retracting of the plunger within the barrel.

Also provided herein is a method of using a system for line draw flushing and blood collection. The method includes providing a syringe assembly including a pre-filled syringe including a barrel having a distal end and a proximal end and that defines a chamber containing a solution therein, and a fluid connector component at the distal end of the barrel. The syringe assembly also includes a plunger assembly including a plunger having a proximal end and a distal end, and a stopper positioned at the distal end of the plunger that is insertable into the proximal end of the barrel and slidable therein, the plunger including a channel extending along a length thereof between the proximal end and the distal end. The syringe assembly also includes an access device positioned at the proximal end of the plunger, the access device comprising a receiving cavity and a fluid access component, the receiving cavity having an opening therein aligned with the channel, and the fluid access component positioned over the opening and extending into the receiving cavity, the fluid access component in fluid communication with the channel. The syringe assembly also includes a cannula positioned at least partially within the barrel and extending through the stopper, with the cannula configured to place the fluid connector component in fluid communication with the channel. The syringe assembly also includes a closure assembly secured to the fluid connector component, the closure assembly comprising a seal member configured to seal off a lumen of the fluid connector component to contain the solution within the chamber of the syringe. The method further includes removing the closure assembly from the fluid connector component, coupling the fluid connector component to an access port of a vascular access device, to place the syringe assembly in fluid connection with the vascular access device, distally advancing the plunger assembly to flush the vascular access device with the solution, obtaining a diversion or discard blood volume via operation of the syringe assembly, and subsequent to obtaining the diversion or discard blood volume, obtaining a sample blood volume via operation of the syringe assembly.

In some embodiments, the closure assembly further includes an end cap configured to engage the fluid connector component, wherein the fluid path seal is positioned within end cap, and a scrubbing cap secured to a distal end of the end cap, the scrubbing cap comprising a housing secured to the end cap and defining a cavity, a scrubbing insert positioned within the cavity, and a seal attached over the cavity to seal the scrubbing insert within the cavity. The method further includes, prior to removing the closure assembly from the fluid connector component, positioning the scrubbing cap over the access port of the vascular access device, such that the scrubbing insert contacts the access port, and cleaning the access port with the scrubbing insert.

In some embodiments, obtaining the diversion or discard blood volume comprises proximally retracting the plunger assembly within the barrel, to draw the diversion or discard blood volume into the chamber of the barrel.

In some embodiments, the method further includes locking the plunger in place relative to the barrel subsequent to proximally retracting the plunger assembly to draw the diversion or discard blood volume into the chamber of the barrel.

In some embodiments, the step of obtaining the diversion or discard blood volume includes positioning a container within the receiving cavity, the container defining an evacuated reservoir therein, engaging the container with the fluid access component to draw the diversion or discard blood volume into the evacuated reservoir, and removing the container from the receiving cavity.

In some embodiments, the step of obtaining the sample blood volume includes positioning a container within the receiving cavity, the container defining an evacuated reservoir therein, engaging the container with the fluid access component to draw the sample blood volume into the evacuated reservoir.

In some embodiments, the fluid access component comprises a needle having a lumen therein, and where engaging the container with the fluid access component comprises piercing a cover of the container with the needle, to position the needle partially within the reservoir.

In some embodiments, the method further includes locking the plunger in place relative to the barrel subsequent to distally advancing the plunger assembly to flush the vascular access device with the solution.

### BRIEF DESCRIPTION OF THE DRAWINGS

FIG. 1 is a perspective view of a system for line draw flushing and blood collection, including a syringe assembly and a collection container, according to an embodiment;

FIG. 2 is a side cross-sectional view of the system of FIG. 1;

FIG. 3 is an exploded view of the system of FIG. 1;

FIGS. 4A-4H illustrate process steps for using the system of FIG. 1, including for the cleaning of and connection to an access connector, according to an embodiment;

FIG. 5 is a side view of a syringe assembly, according to an embodiment;

FIG. 6 is an exploded view of the syringe assembly of FIG. 5;

FIG. 7 is a side view of a syringe assembly, according to an embodiment;

FIG. 8 is an exploded view of the syringe assembly of FIG. 7;

FIG. 9 is a side view of a syringe assembly, according to an embodiment;

FIG. 10 is an exploded view of the syringe assembly of FIG. 9;

FIG. 11 is a side view of a syringe assembly, according to an embodiment;

FIG. 12 is an exploded view of the syringe assembly of FIG. 11;

FIG. 13 is a side view of a syringe assembly, according to an embodiment;

FIG. 14 is an exploded view of the syringe assembly of FIG. 13;

FIG. 15 is a side view of a syringe assembly, according to an embodiment; and

FIG. 16 is an exploded view of the syringe assembly of FIG. 15.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the systems described below may be utilized for blood draw from any suitable vascular access device (VAD) such as, e.g., the BD NEXIVA™ Closed IV Catheter system, the BD CATHENA™ Catheter system, the BD VENFLON™ Pro Safely Shielded IV Catheter system, the BD NEOFLONTM IV Cannula system, the BD INSYTE™ AUTOGUARD™ BC Shielded IV Catheter system, or another suitable vascular access device.

Embodiments of the present disclosure will primarily be described in the context of a device for flushing and blood sample collection for use with PIVCs. However, embodiments of the present disclosure equally extend to use with other catheter devices.

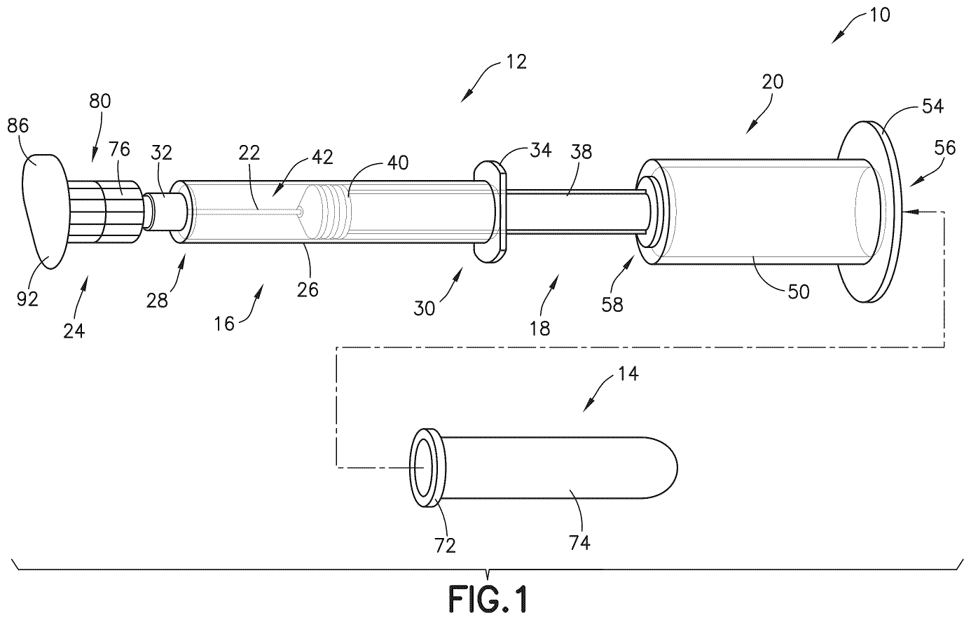

Referring to FIGS. 1-3, a system for line draw flushing and blood collection 10 (hereafter “system 10”) is shown in accordance with an aspect of the present disclosure. The system 10 includes a syringe assembly 12 and a blood collection container 14 useable with the syringe assembly 12. As described in further detail below, the system 10 may be employed by a user (e.g., clinician) to first flush a line draw vascular access device (VAD), then obtain an initial discard sample of blood in the syringe assembly 12 (or a discard collection container), and finally obtain a blood culture sample in the collection container 14 that may be analyzed to detect the presence of bacteria or fungi.

As shown in FIGS. 1-3, the syringe assembly 12 may be generally characterized as including a syringe 16, a plunger assembly 18, an access device 20, a cannula 22, and a closure assembly 24.

The syringe 16 may be structured as a generally cylindrical barrel 26 that includes a distal end 28 and a proximal end 30, with a fluid connector component 32 at the distal end 28 and a finger grip 34 at the proximal end 30. According to embodiments, the fluid connector component 32 may be integrated with or removably coupled to the distal end 28 of the barrel 26. According to embodiments, the fluid connector component 32 may include any suitable component configured to couple the barrel 26 to a connector (e.g., needle-free connector) or access port of a vascular access device (VAD). For example, in some embodiments, the fluid connector component 32 may be a luer connector. The luer connector may be in the form of, e.g., a slip luer, a threaded luer, a luer lock with collar, etc. In some embodiments, the fluid connector component 32 provides an outlet of the barrel 26 via a lumen 36 formed therein.

The plunger assembly 18 includes a plunger 38 and a stopper 40. As shown in FIGS. 1-3, plunger 38 is formed as a generally elongated member that is insertable into a chamber 42 defined by the barrel 26 of syringe 16, with the plunger 38 having a distal end 44 and a proximal end 46. A channel 48 is formed within the plunger 38 extending the length thereof. In one embodiment, the channel 48 is tapered along its length—with a diameter of the channel 48 increasing as the channel 48 extends from the distal end 44 to the proximal end 46.

The stopper 40 of plunger assembly 18 is positioned at the distal end 44 of plunger 38 so as to be movable along with the plunger 38 within the chamber 42 of syringe barrel 26. The stopper 40 may be made from a material that is different from the material of the plunger 38 and that is capable of forming a tight seal with the syringe barrel 26 as it is advanced therethrough. In some embodiments, the stopper 40 includes a receptacle (not shown) formed therein that is sized and configured to receive a threaded connection 49 formed on the distal end 44 of plunger 38, with the threaded connection 49 coupling with the receptacle via a threaded/twist-type connection, for example, to secure the stopper 40 to the plunger 38, although it can be appreciated that the stopper 40 can be secured to the distal end 44 of plunger 38 by other techniques known in the art.

The access device 20 is positioned at the distal end 44 of the plunger 38 and includes a receiving cavity 50, a fluid access component 52, and a finger grip 54. In the illustrated embodiment, the plunger 38, receiving cavity 50, and finger grip 54 are a single integral component. For example, the plunger 38, receiving cavity 50 and finger grip 54 may be formed as a molded component.

The receiving cavity 50 is shaped to generally form a receptacle within which a container 14 may be positioned, with the receiving cavity having an open proximal end 56 for receiving the container 14. A distal end 58 of the receiving cavity 50 is joined to (e.g., formed with) the plunger 28 and includes an opening 60 formed therein that is aligned with channel 48. A plunger seal 62 is positioned within the opening 60 to separate the channel 48 from the receptacle of the receiving cavity 50, and the fluid access component 52 is positioned within the opening 60 and extends through the plunger seal 62. The fluid access component 52 may be secured within plunger seal 62 and extend out therefrom into the interior of the receiving cavity 50. In one embodiment, the fluid access component 52 may include a needle 64 defining a lumen therein. In some embodiments, a flexible needle sheath 66 may be provided over needle 64 that is configured to be translated relative to the needle 64, such that a proximal end of the needle 64 may be selectively exposed.

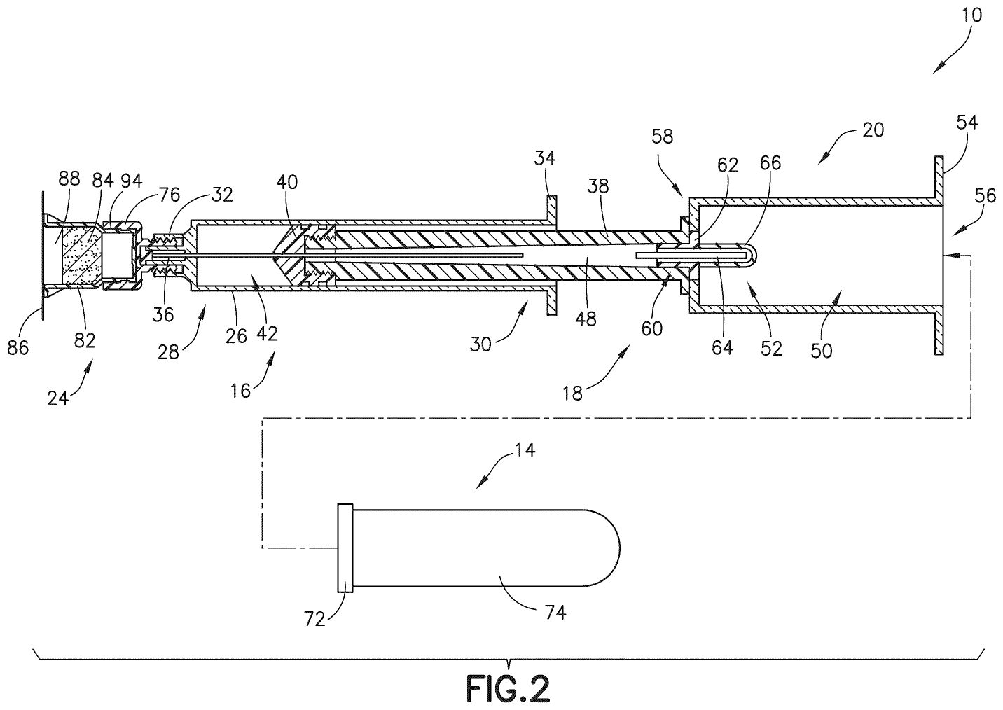

Referring still to FIGS. 1-3, it is shown that cannula 22 is incorporated into syringe assembly 12 via positioning thereof within syringe 16 and plunger assembly 18. In one embodiment, the cannula 22 is provided in syringe assembly 12 so as to be affixed to the barrel 26 and such that a distal end 68 thereof is positioned substantially flush with an opening 70 in fluid connector component 32 of syringe 16, with the cannula 22 extending proximally from the fluid connector component 32, through a portion of chamber 42 of barrel 26 and through a hole formed in stopper 40, and into the channel 48 formed in plunger 38, as best shown in FIG. 2. In some embodiments, the cannula 22 may be recessed rearward of the opening 70 in the fluid connector component 32, with this recessed arrangement providing the syringe assembly 12 compatibility with needleless connectors, luer-activated valves and/or IV connectors that may have an internal spike or similar projection intended to insert into the fluid connector component 32 for functionality.

According to aspects of the disclosure, the cannula 22 has a cross-section smaller than the opening 70 at the tip of fluid connector component 32, allowing fluid to flow through a gap between the outer surface of the cannula 22 and the inner surface of the opening 56. According to embodiments, the cannula 22 is secured to fluid connector component 32 so as to not impede the flow of fluids between the gap. In one embodiment, the cannula 22 is secured to the fluid connector component 32 via one or more retaining features of the fluid connector component 32, which may comprise flanges, struts, wings, a beveled flange, or other mechanical structures (not shown) extending between the inner surface of the fluid connector component 32 and the outer surface of the cannula 22. In other embodiments, the cannula 22 may be secured to the fluid connector component 32 using an adhesive, thermal bonding, or other suitable means.

With cannula 22 sized and positioned relative to syringe 16 and plunger assembly 18 as described above, separate fluid paths are provided in syringe assembly 12 via which blood may be drawn into system 10. A (first) fluid path is formed via the gap between the outer surface of the cannula 22 and the inner surface of the opening 70 through which fluid (i.e., blood) may flow from fluid connector component 32 and into a portion of chamber 42 within barrel 26—i.e., a fluid chamber defined by a distal surface of the stopper 40 and an inner surface of the distal end 28 of barrel 26. A (second) fluid path is provided via the cannula 22 through which fluid (i.e., blood) may flow from fluid connector component 32 and into the channel 48 of plunger 38, with the fluid then being transferrable out from channel 48 and through fluid access component 52 into a container 14.

Referring again to FIG. 1, the container 14 may have any of a number of structures that enable the container 14 to be inserted into receiving cavity 50 (defined by a cylindrical wall structure 64), with FIG. 1 illustrating container 14 as a tube, such as a BD VACUTAINER® from Becton, Dickinson and Co., as a non-limiting example, but it recognized that container 14 could be provided as a necked vial or other suitable receptacle. The container 14 includes a cover 72 and a fluid reservoir 74, with the cover 72 disposed at a distal end of the container 14. In some embodiments, the cover 72 may include a resealable membrane configured such that a fluid access component, such as needle 64 of fluid access component 52, can pierce the resealable membrane to achieve fluidic communication with the reservoir 74. The resealable membrane of the cover 72 may be configured to reseal upon decoupling the fluid access component 52 from the cover 72 such that the reservoir 74 is fluidically isolated from an area external to the container 14. The reservoir 74 can be an evacuated reservoir such that, upon the reservoir 74 being placed in fluidic communication with a source of fluid (e.g., via piercing the resealable membrane of the cover 72 with a needle 62 fluidically coupled to a patient's vasculature), fluid (e.g., blood) can be drawn into the reservoir 74 due to a pressure differential between the reservoir 74 and the source of fluid.

As shown in FIGS. 1-3, closure assembly 24 is secured to the fluid connector component 32 of syringe assembly 12. The closure assembly 24 includes an end cap 76 configured to engage the fluid connector component 32, so as to cover the fluid connector component 32 when syringe assembly 12 is not in use. In one embodiment, a proximal end of the end cap 76 is configured as a luer fitting configured to mate with the associated luer fitting of the fluid connector component 32, so as to secure the end cap 76 to syringe 16. The closure assembly 24 also includes a seal member 78 configured to seal off the lumen 36 of the fluid connector component 52. As best shown in FIG. 2, seal member 78 may be positioned/secured within an interior cavity of end cap 76, such that when the end cap 76 is secured to the fluid connector component 32, the seal member 78 is inserted into the opening 70 at the tip of fluid connector component 32, thereby sealing the lumen 36.

According to aspects of the disclosure, it is recognized that when initially connecting the system 10 to a VAD access port, such as via coupling of the fluid connector component 32 with the access port, it is desirable to disinfect the access port prior to connection of system 10 thereto. That is, it is desirable to clean and sterilize the access port prior to engagement of fluid connector component 32 therewith (e.g., via mating of female and male luer connections) to prevent microbial ingress and possible catheter-related blood stream infections (CRBSIs).

Accordingly, some embodiments of syringe assembly 12 include, a scrubbing cap 80 that is integrated into the closure assembly 24 with the syringe assembly 12 that provides for cleaning and sterilizing of the access port prior to engagement of the system 10 therewith. The scrubbing cap 80 is secured onto a distal end of end cap 76 and is configured to engage with a VAD access port to enable the cleaning and sterilizing of internal and/or external surfaces thereof, such as via a twisting and scrub motion between the scrubbing cap 80 and access port. The closure assembly 24 may be removed from fluid connector component 32 upon completion of the cleaning and sterilizing of the access port, thereby enabling a subsequent coupling of fluid connector component 32 to the access port and performing of a blood draw via operation of system 10 as previously described.

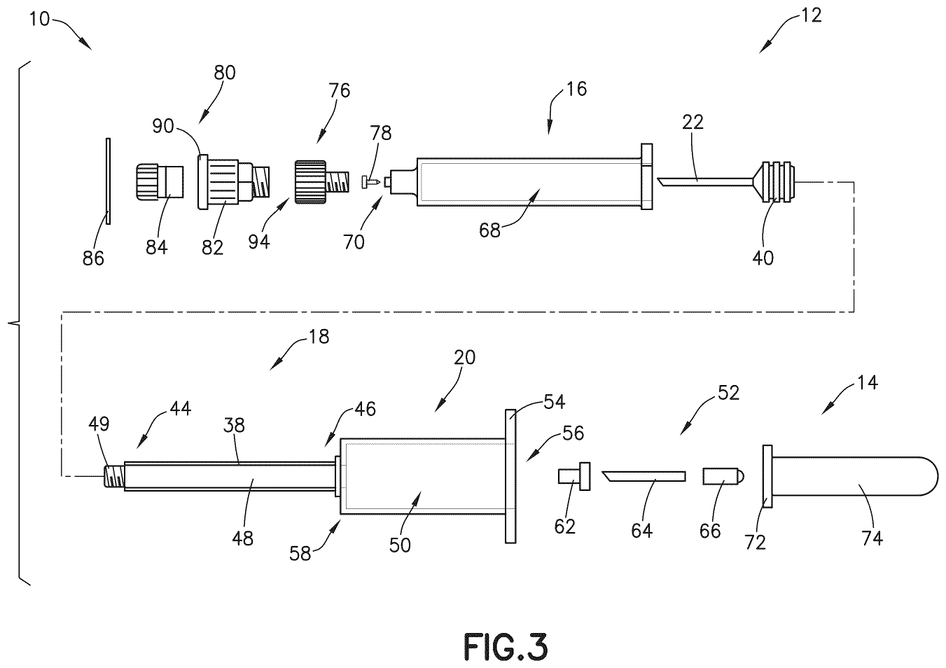

As best shown in FIGS. 2 and 3, scrubbing cap 80 includes a cap housing 82, scrubbing insert 84, and a pealable seal 86. The housing 82 may be made by injection molding and may be made of an alcohol compatible material, such as polypropylene or polyethylene, for instance. The housing 82 may have a cup shape that defines an open cavity 88 configured to receive the insert 84 therein. In some embodiments, the cavity 88 may have a cylindrical cross-sectional shape and may have a depth suitable to receive the insert 84 therein in a recessed manner. However, it is appreciated that the housing 82 and the cavity 88 defined thereby can assume other shapes, including square, hexagonal, etc., and that the housing 82, its cavity 88, and the insert 84 disposed therein can be configured in shape and size so as to enable the scrubbing cap 80 to cleanse a particular size and configuration of a medical device with which the scrubbing cap 80 is to be employed (i.e., catheter connector 20 of catheter assembly 12).

The housing 82 may be sized such that the cavity 88 compresses the insert 84 when the insert 84 is fitted into the cavity 88, so as to retain the insert 84 therein. A suitable hot melt glue or other suitable adhesive may also be used to adhere the insert 84 to the bottom of the housing 82 (i.e., to base 82), although it is recognized that other suitable methods can also be employed to secure the insert 84 to the housing 82, including mechanical fixation for instance. An annular lip 90 may be formed on the housing 82 to define a land for receiving the seal 86, with the seal 86 cooperating with the annular lip 90 to seal the cavity 88 and retain the insert 84 therein. The seal 86 seals the cavity 88 of the housing 82 and the insert 84 therein against contamination from the outside environment and provides a leak-proof barrier, thereby protecting the contents of insert 84 and maintaining a sealed, sterilized environment. The seal 86 provides a sufficient seal at a range of temperatures, pressures, and humidity levels and, according to embodiments, may be formed as an aluminum or multi-layer polymer film peel back top. In some embodiments, the seal 86 is heat-sealed or induction sealed to the open end of the scrubbing cap 80. The seal 86 can include a tab 92 to facilitate the manipulation of and removal of the seal 86 from the scrubbing cap 80.

The insert 84 is constructed of a foam material, for example, of injection molded construction or the insert 84 may be die-cut from a foam sheet. The insert includes a cleansing substance impregnated therein (while in the housing 82), such as a solution of a suitable microbiocide or germicide. The cleansing substance can include an anti-bacterial disinfectant of any suitable type and suitable amount depending upon the size of the insert of foam material. For example, in one embodiment use is made of an aqueous solution including about two percent (2%) chlorhexidine gluconate (chlorhexidine solution, “CHG”) by volume in an amount of from about 0.20 cc to about 0.75 cc. Optionally, a solution including about 0.50 cc is employed. In another embodiment, a solution including about 70 percent (70%) isopropyl alcohol (“IPA”) in an aqueous solution is included in the cleansing substance. In yet another embodiment, a solution including about 70 percent (70%) IPA and about two percent (2%) CHG in an aqueous solution in an amount of about 0.2 ml is included in the cleansing substance. In the latter solution, it is recognized that the concentration of IPA can vary from about 60 percent (60%) to about 90 percent (90%) and the concentration of CHG can vary from about one percent (1%) to about five percent (5%), in one embodiment. Other suitable solution compositions and concentrations are also possible. For instance, povidone iodine or hydrogen peroxide solutions can be included in the cleansing substance, in one embodiment.

According to aspects of the disclosure, the insert 84 may be constructed (e.g., molded) to have a predefined shape that conforms to the unique shape of the VAD access port or connector that is to be cleaned thereby. The insert 84 may further have any of a number of constructions that provide for effective cleaning of the port/connector, such as including a patterned or roughened top surface and/or gaps or slits formed in the foam material thereof that enables the insert 84 to better deform/conform about interior and exterior surfaces of the port/connector.

The housing 82 of scrubbing cap 80 has a proximal end configured to mate with end cap 76 to secure the scrubbing cap 80 thereto. According to embodiments, the housing 82 may connect with end cap 76 via insertion of the housing into receptacle 94 provided on the distal end of end cap 76, with the scrubbing cap 80 secured to the end cap 76 via a press-fit connection between the housing 82 and the receptacle 94, a threading engagement between the housing 82 and the receptacle 94, or any other suitable engagement means that secures the housing 82 is in place relative to end cap 76.

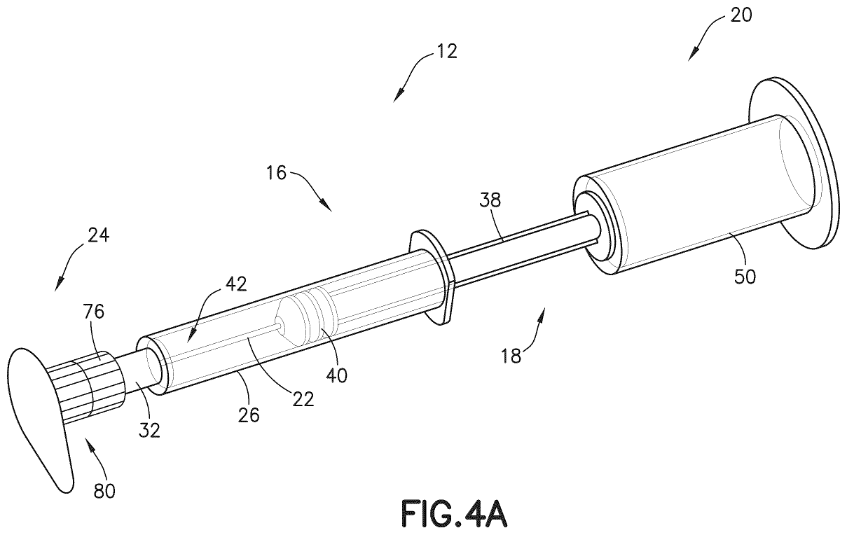

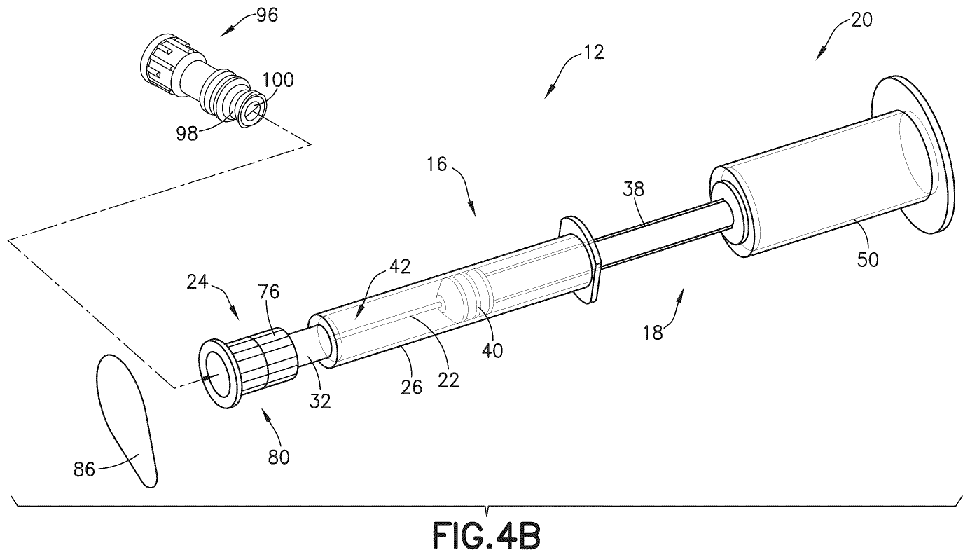

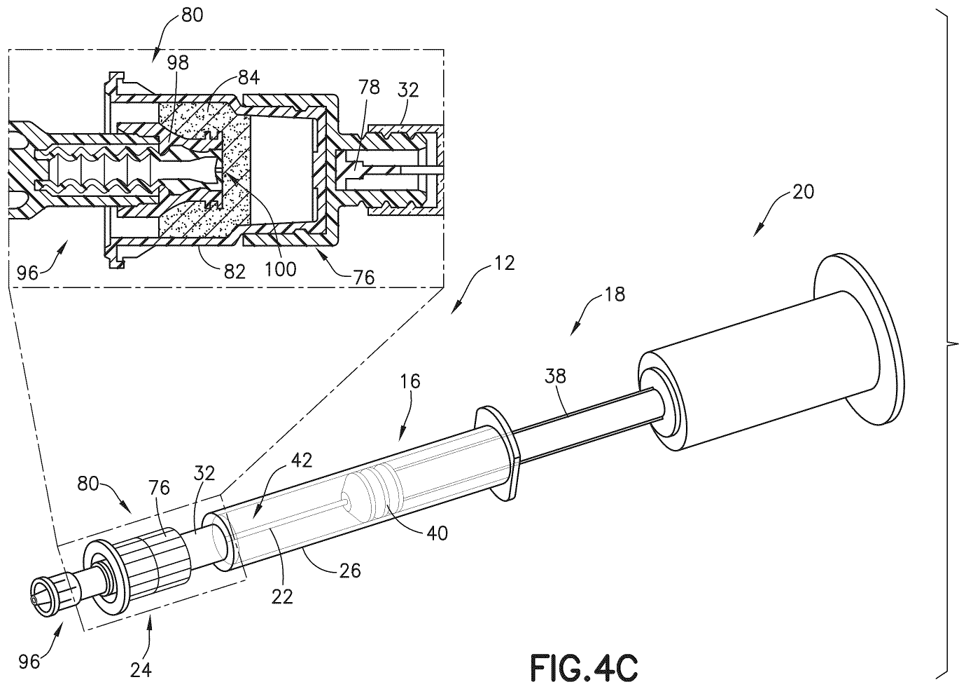

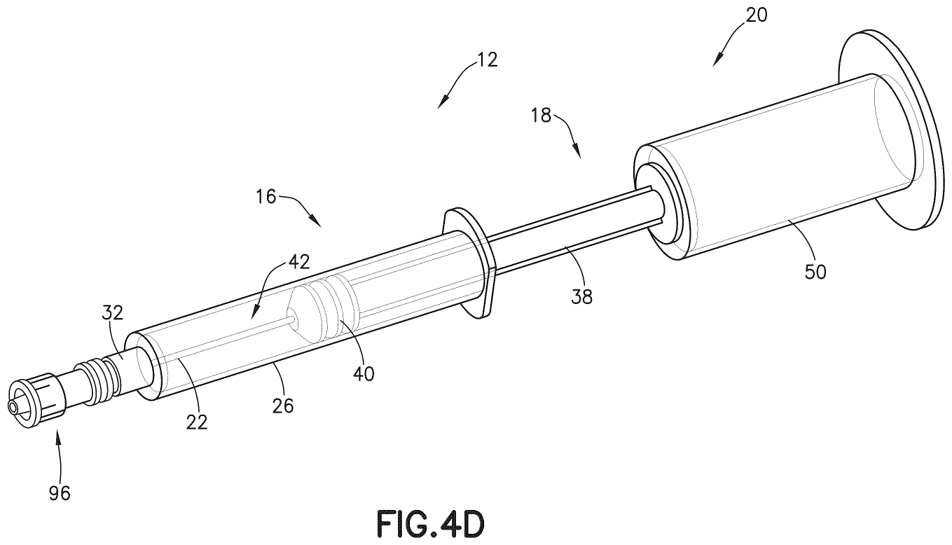

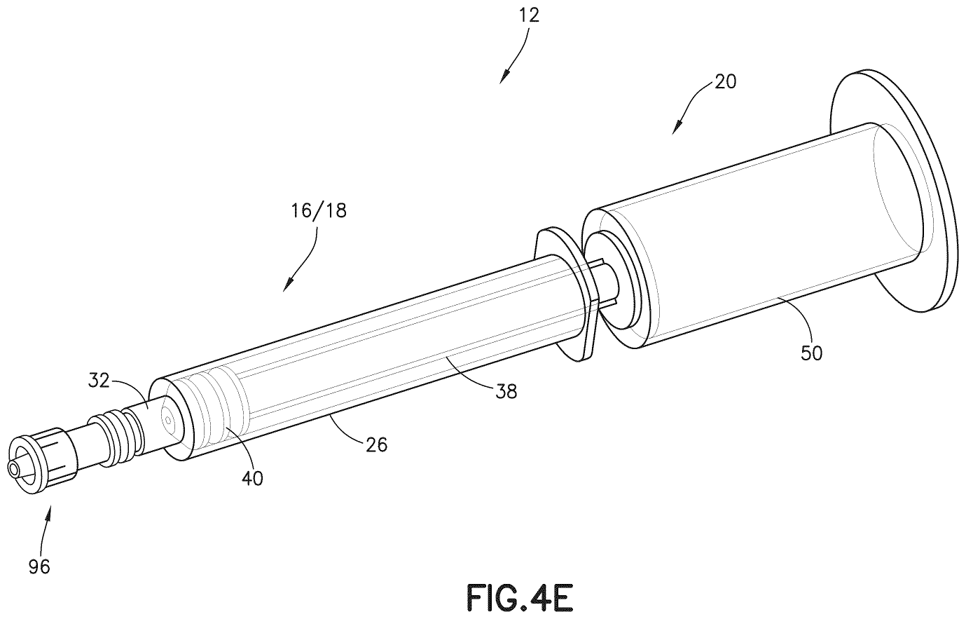

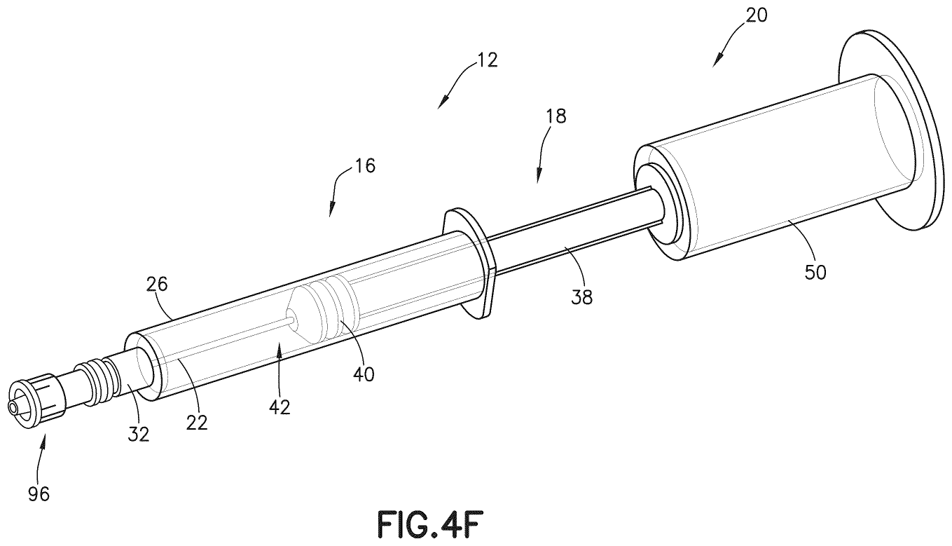

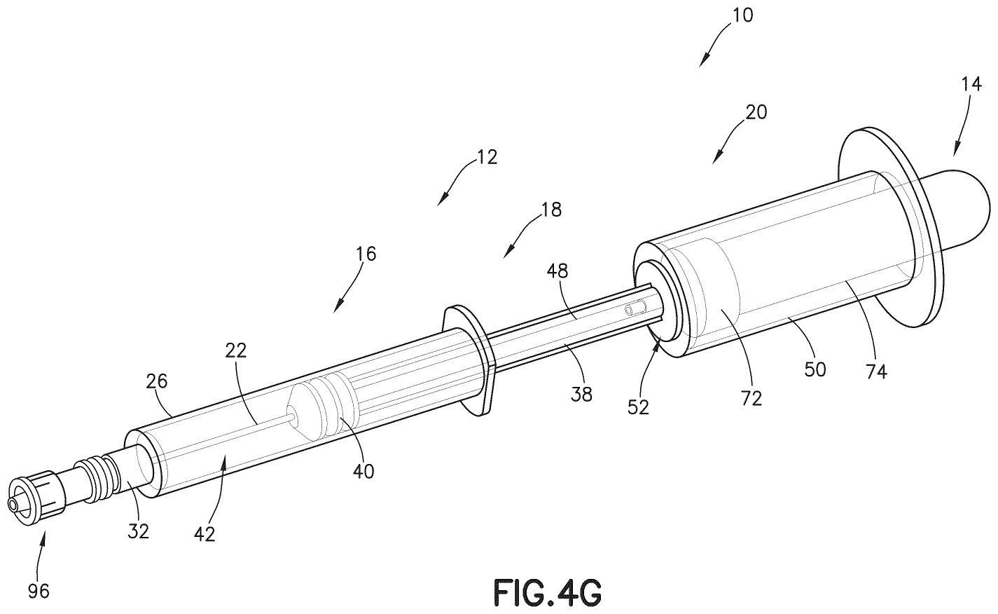

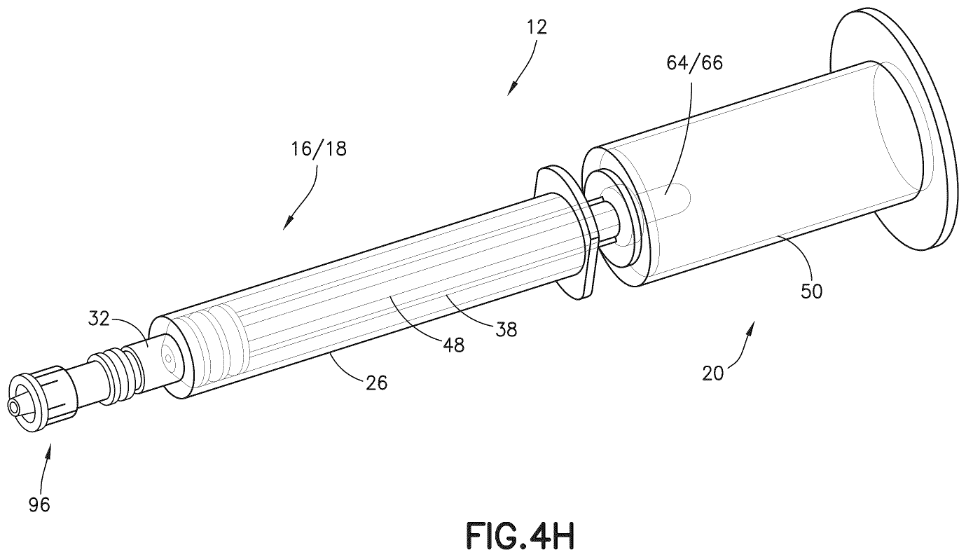

Following here below, and as shown in FIGS. 4A-4H, use of a system 10 for line draw flushing and blood collection is described in accordance with one aspect of the disclosure. Use of the system 10 begins with the syringe assembly 12 in an initial configuration as shown in FIG. 4A. The syringe assembly 12 is provided as a pre-filled syringe that, in the initial configuration, contains a solution therein (i.e., contained in barrel 26) for flushing the fluid path/line of a VAD to which the syringe assembly 12 is to be connected. When in the initial configuration, the plunger 38 is provided in an intermediate position that is neither fully advanced nor fully retracted, such that a fluid chamber is defined within barrel 26 (by an inner surface of the distal end 28 of the barrel 26 and the distal surface of stopper 40) holding a desired volume of flushing solution. As known in the art, the flushing solution may be 0.9% sodium chloride injection solution, saline solution, or a heparin solution, as non-limiting examples. As also shown in FIG. 4A, when in the initial configuration, the closure assembly 24 is secured on fluid connector component 32 of syringe 16—with end cap 76 and scrubbing cap 80 secured to the fluid connector component 32 of syringe 16 and the scrubbing cap 80 in a sealed condition (i.e., seal 86 is secured to housing 82, so as to seal-off scrubbing insert 84).

Use of the system 10 continues as shown in FIGS. 4B and 4C by removing the seal 86 from the scrubbing cap 80 of syringe assembly 12 in order to provide access to the scrubbing insert 84. Upon removal of the seal 86, the syringe assembly 12 is brought into proximity to a VAD (not shown) to provide for cleaning and sterilization of an access port or connector 96 of the VAD, with FIGS. 4B and 4C illustrating a needle-free connector according to a non-limiting example. Upon the syringe assembly 12 brought into proximity to the connector 96, the scrubbing cap 80 is positioned over a portion of the connector 96 (i.e., a female luer connection having a threaded outer connection 98 defining a tapered lumen or cavity 100, according to a non-limiting embodiment), with the foamed insert 84 pushed onto the connector 96 by the user. At this time, portions of the insert 84 conform about and/or are inserted into the connector 96 (i.e., about threaded outer connection 98 and into lumen 100). Once the portion of the connector 96 has been inserted into the foamed insert 84 of the scrubbing cap 80, the scrubbing cap 80 is rotated relative to the connector 96. For example, the user may hold the connector 96 stationary while rotating the syringe assembly 12. As the scrubbing cap 80 is secured in place relative to the syringe 16—via the connection of fluid connector component 32 to end cap 76 and scrubbing cap 80—rotation of the syringe assembly 12 causes a corresponding rotation of scrubbing cap 80. Rotation of the scrubbing cap 80 relative to the connector 96 may be done all in one direction or may be a back-and-forth twisting motion. The scrubbing cap 80 is rotated a sufficient number of times relative to the connector 96 to sufficiently kill any bacteria that the solution-impregnated foam insert 84 comes in contact with and/or to remove any biofilm from the outside peripheral surface and threaded outer connection 98 of connector 96 as well as the inside surface of the lumen 100 of the connector 96. In this way, both exterior surfaces and interior luminal surfaces of the connector 96 are scrubbed by the insert 84, causing the cleansing substance therein to disinfect the surfaces and remove any biofilm disposed thereon.

Upon completion of the cleaning and sterilizing of the connector 96 of the VAD via scrubbing cap 80, the scrubbing cap 80 is separated away from connector 96. The closure assembly 24 (scrubbing cap 80, end cap 76, and seal member 78) may then be removed from fluid connector component 32 of syringe assembly 12. After allowing the connector 96 to dry, the syringe assembly 12 may then be connected to the VAD by coupling the fluid connector component 32 to connector 96, as shown in FIG. 4D, thereby fluidly connecting syringe assembly 12 to catheter assembly 100.

According to an aspect of the disclosure, upon connection of syringe assembly 12 to connector 96, the connector 96 and a fluid path of the VAD may be flushed with the flushing solution provided in the pre-filled syringe 16. As shown in FIG. 4E, the plunger assembly 18 (i.e., plunger 38 and stopper 40) is advanced distally to force the flushing solution out of barrel 26 through lumen 36 of fluid connector component 32 and into the VAD fluid path. The plunger 38 and stopper 40 may be actuated to their fully advanced position to empty the flushing solution from syringe 16.

After flushing of the VAD fluid path is complete, the syringe assembly 12 may then be operated to collect an initial discard sample of blood. As shown in FIG. 4F, the plunger assembly 18 (i.e., plunger 38 and stopper 40) is retracted proximally to draw an initial discard sample of blood into syringe assembly 12. Upon the plunger 38 and stopper 40 being retracted proximally within syringe barrel 26, a discard (or diversion) volume of blood is drawn into a portion of chamber 42—i.e., a fluid chamber defined by a distal surface of the stopper 40 and an inner surface of the distal end 28 of barrel 26—with blood flowing along a first fluid path from fluid connector component 32 and into the portion of chamber 42.

Upon drawing of a sufficient discard sample of blood, the system 10 may then subsequently be operated to acquire a desired blood culture sample within a collection container 14, as shown in FIG. 4G. For acquiring such a sample, a collection container 14 is engaged with syringe assembly 12—with the collection container 14 (e.g., vacutainer) positioned within the receiving cavity 50 and engaged with the fluid access component 52 to place the container 14 in fluid communication with the plunger assembly 18. That is, upon positioning/connection of the collection container 14 within receiving cavity 50 and onto fluid access component 52, a second fluid path is formed from the fluid connector component 32 to the collection container 14—with cannula 22 transferring blood from connector component 32 into channel 48 within plunger 38, and blood then passing from channel 48 through fluid access component 52 and into container 14. As described above, in some embodiments, fluid access component 52 is provided as a needle 64 with a lumen formed therein, with the needle 64 piercing the cover 72 of container 14 so that blood is drawn into the evacuated reservoir 74 within the container 14.

As shown in FIG. 4H, upon acquisition of a blood culture sample within collection container 14, collection container 14 may be removed from syringe assembly 12. Additionally, in some embodiments, the syringe assembly 12 may then be operated to return the discard sample of blood back to the patient. That is, as shown in FIG. 4H, the plunger assembly 18 (i.e., plunger 38 and stopper 40) is advanced distally to force the discard sample of blood out of barrel 26 through lumen 36 of fluid connector component 32 and into the VAD fluid path. The plunger 38 and stopper 40 may be actuated to their fully advanced position to empty the discard sample of blood from syringe 16.

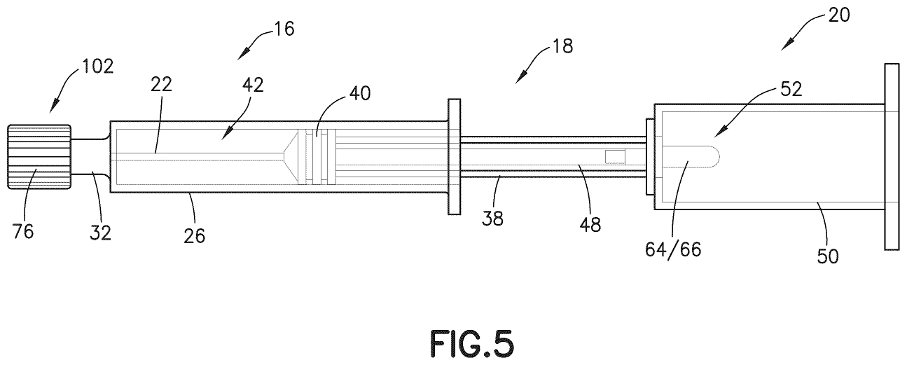

While system 10 and a method of use thereof is shown and described in FIGS. 1-4 according to a specific embodiment, it is recognized that the system 10 (and the syringe assembly 12 thereof) may have other suitable configurations. FIGS. 5-16 illustrate additional embodiments of a syringe assembly that may be used for line draw flushing and blood collection, in accordance with further aspects of the disclosure.

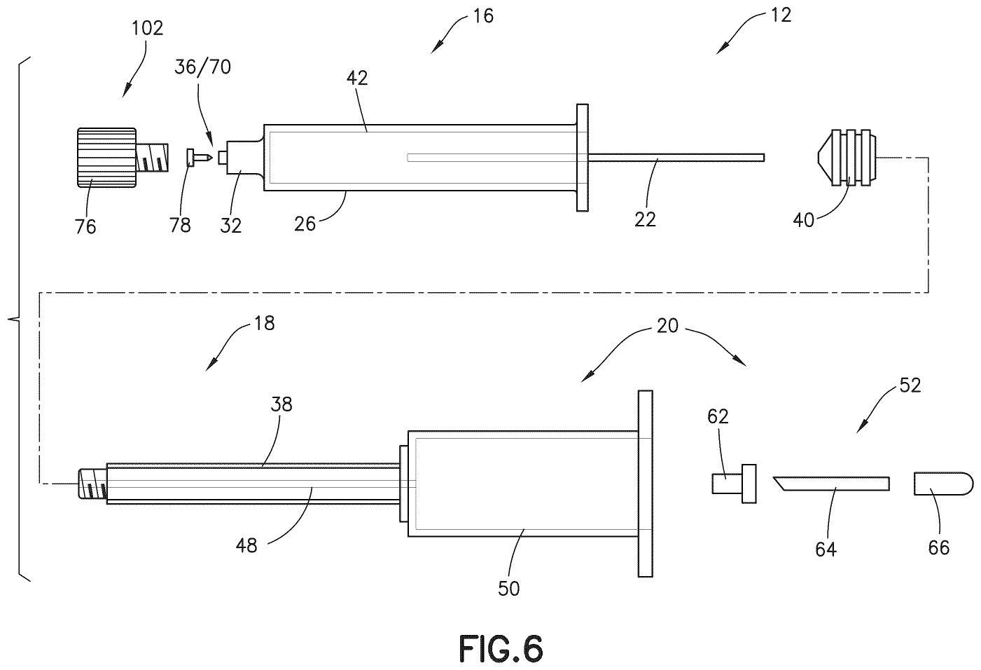

Referring now to FIGS. 5 and 6, a syringe assembly 12 is shown in accordance with another aspect of the disclosure. The syringe assembly 12 includes all the elements of the syringe assembly 12 previously shown and described in FIGS. 1-4, except that a closure assembly 102 included in the syringe assembly 12 does not include a scrubbing cap 80 thereon. Instead, closure assembly 102 includes only end cap 76 and seal member 78. As previously described, end cap 76 is configured to engage the fluid connector component 32, so as to cover the fluid connector component 32 when syringe assembly 12 is not in use, with one embodiment having end cap 76 configured to include a luer fitting at the proximal end thereof configured to mate with the associated luer fitting of the fluid connector component 32. The seal member 78 is positioned/secured within an interior cavity of end cap 76, such that when the end cap 76 is secured to the fluid connector component 32, the seal member 78 is inserted into the opening 70 at the tip of fluid connector component 32, thereby sealing the lumen 36. In closure assembly 102, a distal end of the end cap 76 may present a flat surface or a recessed cavity, with it understood that the end cap 76 covers and closes-off the fluid connector component 32 to maintain the cleanliness thereof.

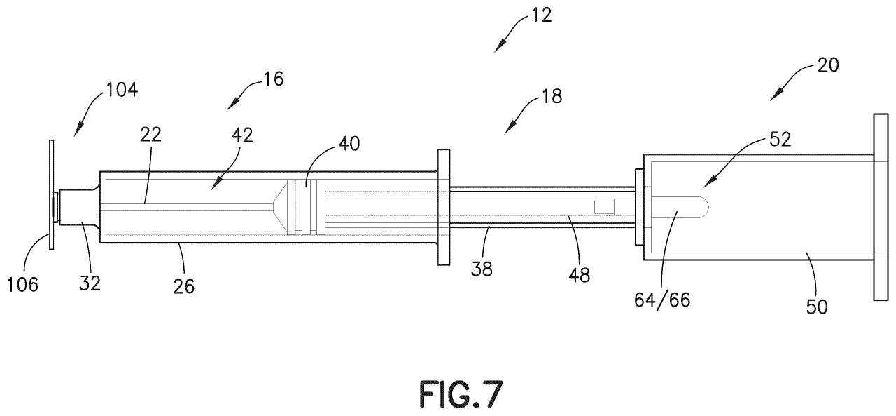

Referring now to FIGS. 7-10, syringe assemblies 12 are shown in accordance with further aspects of the disclosure. In each of the illustrated embodiments, the syringe assembly 12 includes all the elements of the syringe assembly 12 previously shown and described in FIGS. 1-4, except that a closure assembly 104 included in the syringe assembly 12 does not include a scrubbing cap 80 or end cap 76 thereon. Instead, the closure assembly 104 includes only seal member 78 and a peel-off top web 106. The peel-off top web 106 is secured over fluid connector component 32 and to a distal surface of the seal member 78—to a (flat) portion thereof that extends out from opening 70 (and luer 36) at the tip of fluid connector component 32. The peel-off top web 106 may be heat sealed to the distal surface of seal member 78. When it is desired to engage syringe assembly 12 with the connector of a VAD, a user may peel off the top web 106—with removal of the top web 106 also removing seal member 78 from fluid connector component 32 (via the attachment of the top layer 98 and seal member 78) or allowing for a subsequent removal of the seal member 78 from fluid connector component 32.

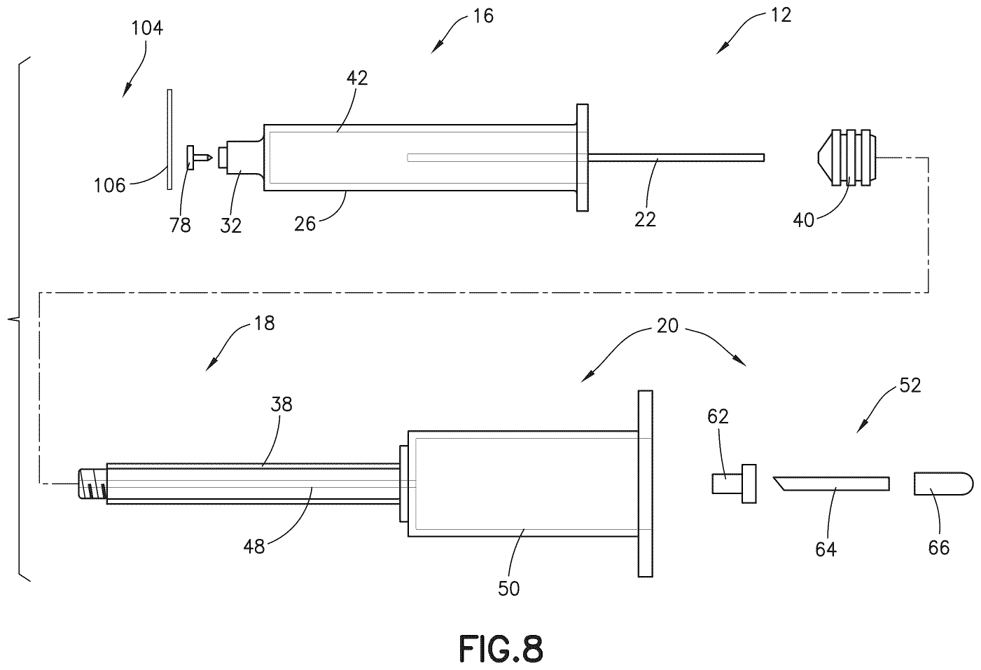

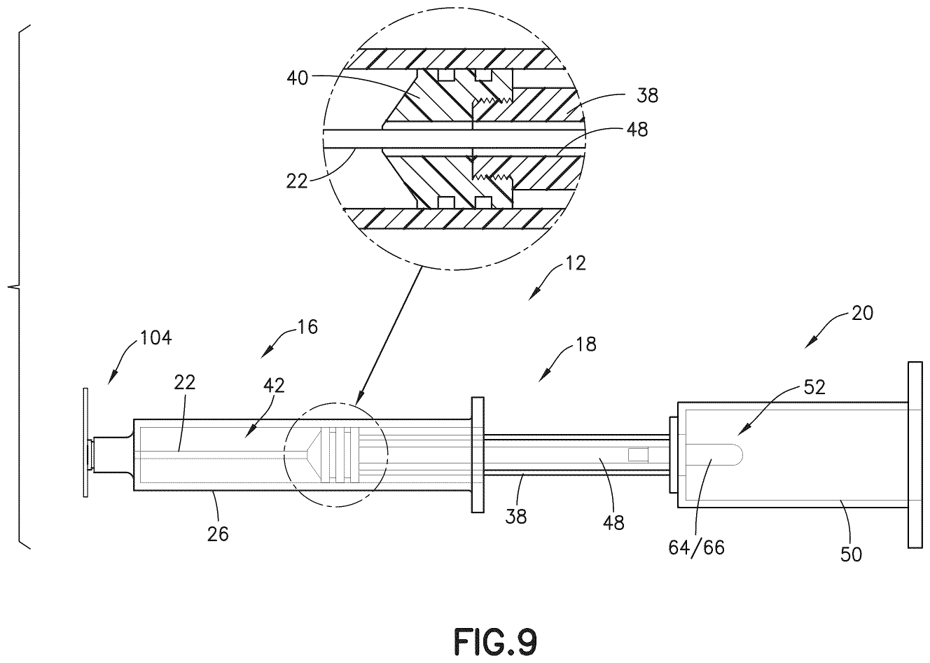

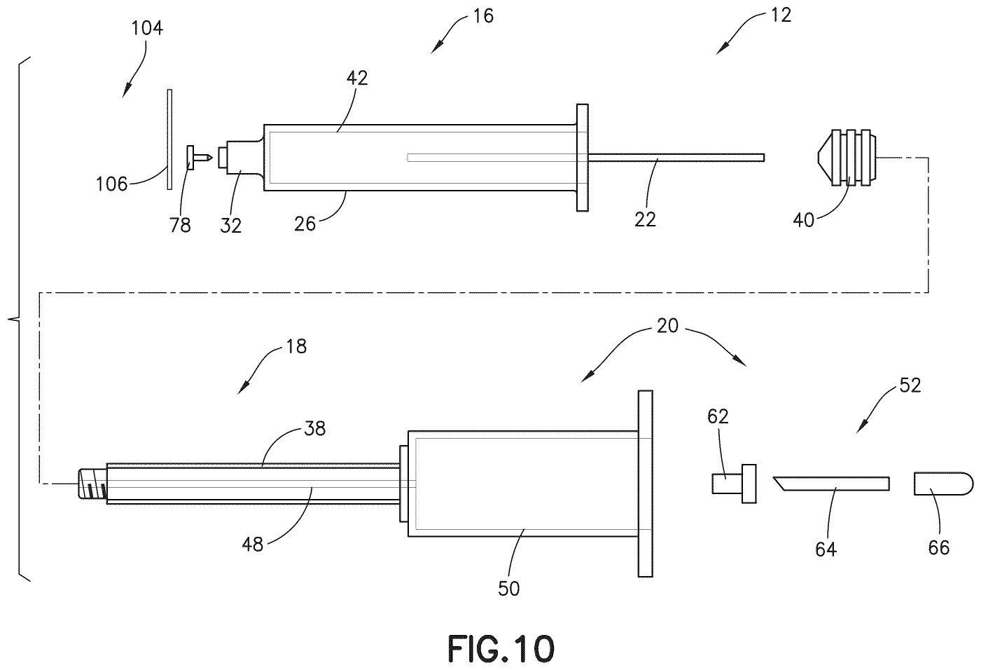

In the syringe assembly 12 shown in FIGS. 7 and 8, the cannula 22 is provided in syringe assembly 12 so as to be affixed to the syringe barrel 26. With the cannula 22 coupled to barrel 26, the cannula 22 thus remains stationary during use of the syringe assembly 12—i.e., during advancing and/or retracting of the plunger assembly 18. In the syringe assembly 12 shown in FIGS. 9 and 10, the cannula 22 is provided in syringe assembly 12 so as to be affixed/bonded to the plunger 38. With the cannula 22 coupled to plunger, the cannula 22 thus moves along with the plunger 38 during use of the syringe assembly 12—i.e., during advancing and/or retracting of the plunger assembly 18.

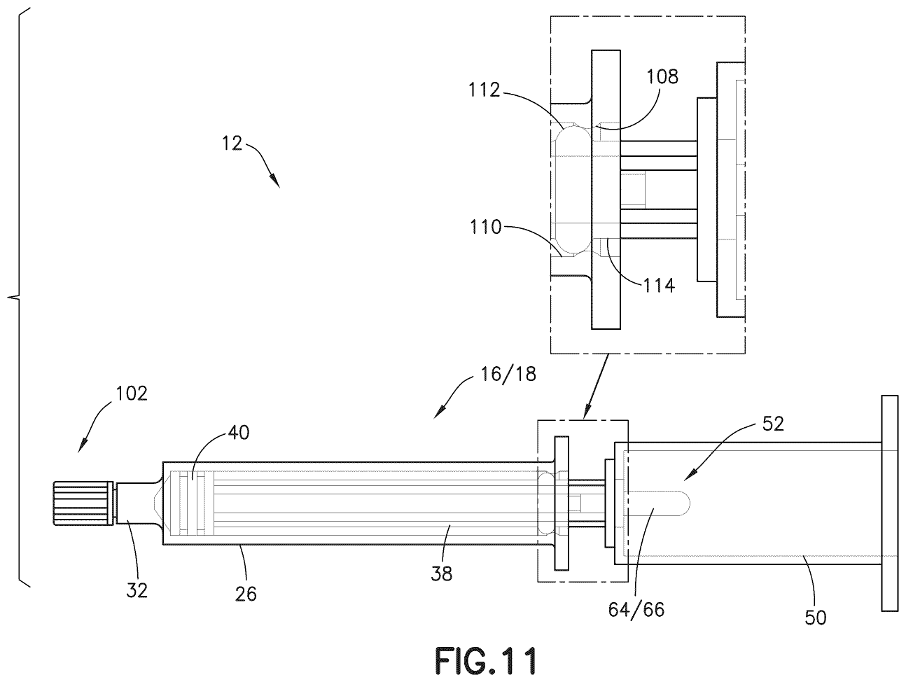

Referring now to FIGS. 11-14, syringe assemblies 12 are shown in accordance with further aspects of the disclosure. In each of the illustrated embodiments, the syringe assembly 12 includes all the elements of the syringe assembly 12 previously shown and described in FIGS. 5 and 6, including a closure assembly 102 that consists of end cap 76 and seal member 78 (with no scrubbing cap 80 thereon). However, each of the syringe assemblies 12 of FIGS. 11-14 further includes therein features that provide for locking of the plunger assembly 18 (i.e., of plunger 38) relative to syringe 16 (i.e., to syringe barrel 26).

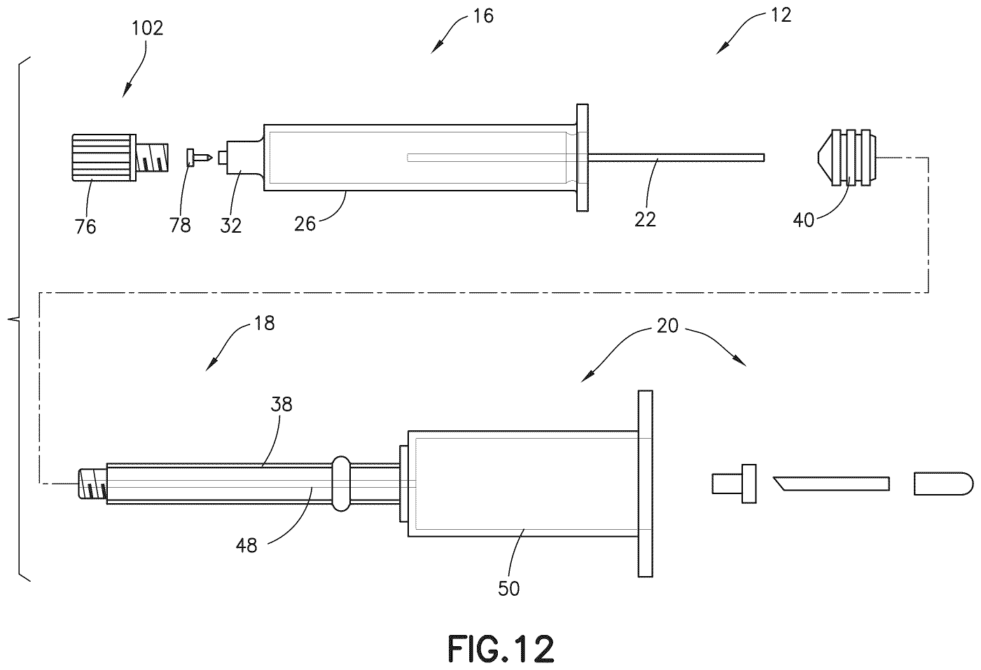

In the syringe assembly 12 shown in FIGS. 11 and 12, the syringe assembly 12 is configured to lock the plunger 38 relative to the syringe barrel 26 when the plunger assembly 18 is in a distally advanced position. To provide for such locking of the plunger 38, the barrel 26 includes a retaining ring 108 formed on an inner surface 110 thereof that extends radially inward from a remainder of the inner surface 110—with the retaining ring 108 of barrel positioned at the proximal end 30 of the barrel 26. The plunger 38 includes a corresponding retaining ring 112 formed thereon that protrudes radially outward from the outer surface 114 of the plunger 38. The retaining ring 112 is formed on plunger 38 adjacent the proximal end 46 thereof. With the retaining rings 108, 112 positioned as such, the retaining ring 112 on plunger 38 can be advanced distally past the retaining ring 108 of barrel 26 when the syringe assembly 18 is fully distally advanced relative to syringe 16 (e.g., with the retaining ring 112 sliding past retaining ring 108). When the retaining ring 112 on plunger 38 is advanced distally past the retaining ring 108 of barrel 26, such as when dispensing/injecting a flushing solution from the syringe assembly 12 into a VAD fluid line, the plunger 38 is then locked within barrel 26 and is prevented from being proximally retracted therefrom.

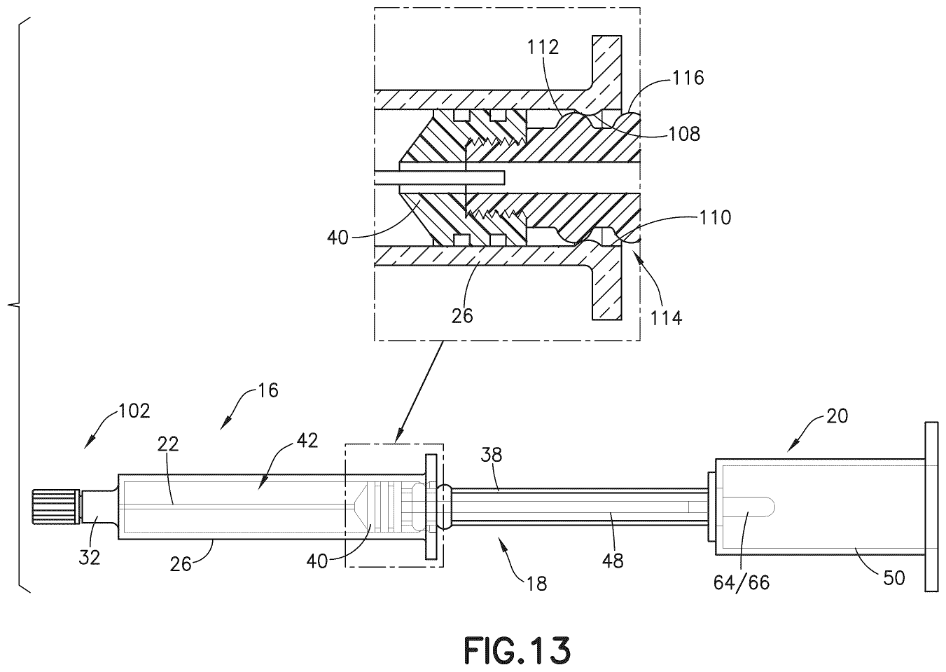

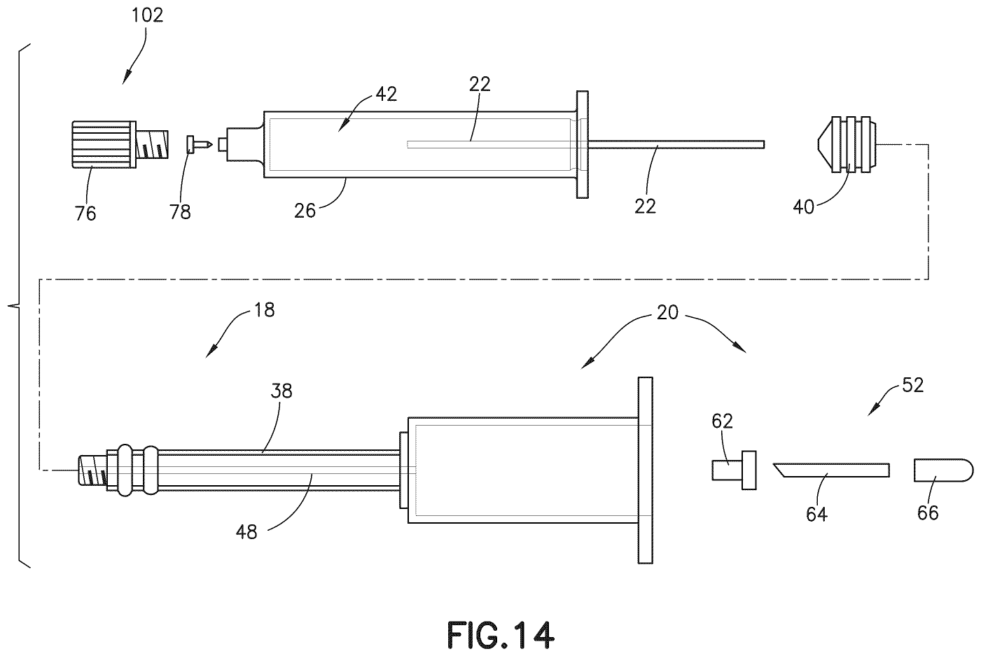

In the syringe assembly 12 shown in FIGS. 13 and 14, the syringe assembly 12 is configured to lock the plunger 38 relative to the syringe barrel 26 when the plunger assembly 18 is in a proximally retracted position. To provide for such locking of the plunger 38, the barrel 26 includes a retaining ring 108 formed on an inner surface 110 thereof that extends radially inward from a remainder of the inner surface 110—with the retaining ring 108 of barrel positioned at the proximal end 30 of the barrel 26. The plunger 38 includes a corresponding pair of retaining rings 112, 116 formed thereon that protrude radially outward from the outer surface 114 of the plunger 38. The retaining rings 112, 116 are formed on plunger 38 adjacent the distal end 44 thereof (e.g., just proximal of stopper 40) in a spaced apart manner. With the retaining rings 108, 112, 116 positioned as such, the plunger 38 may be fully advanced distally into barrel 26 and subsequently proximally retracted back through barrel 26, until the retaining rings 112, 116 on plunger 38 are brought into contact with the retaining ring 108 of barrel 26 (i.e., the retaining rings 112, 116 are brought into position on opposing sides of retaining ring 108). When the retaining rings 112, 116 on plunger 38 are positioned on either side of the retaining ring 108 of barrel 26, such as upon having retracted plunger assembly 18 to aspirate a discard volume of blood into chamber 42 of barrel 26, the plunger 38 is then locked relative to barrel 26 and is prevented from being further proximally retracted or distally advanced.

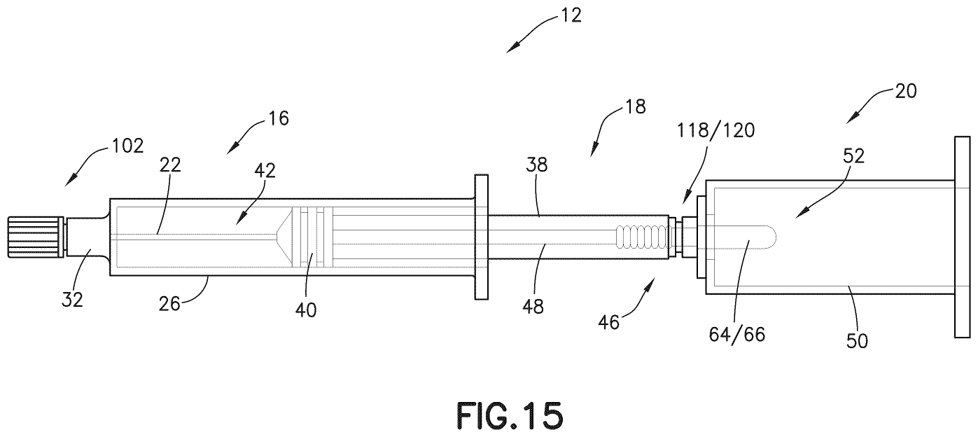

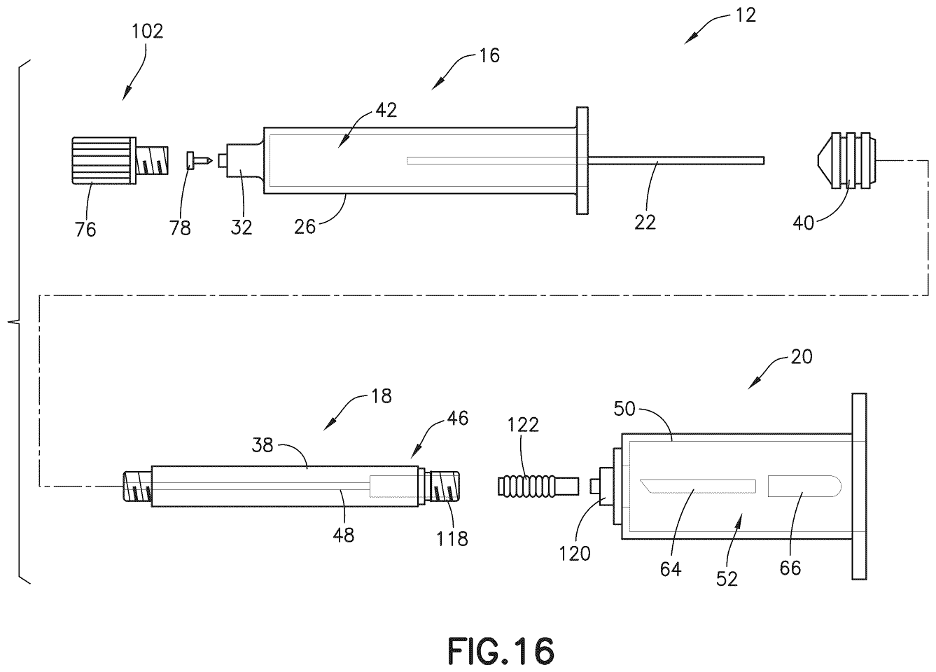

Referring now to FIGS. 15 and 16, a syringe assembly 12 is shown in accordance with another aspect of the disclosure. The syringe assembly 12 includes all the elements of the syringe assembly 12 previously shown and described in FIGS. 5 and 6, including a closure assembly 102 that consists of end cap 76 and seal member 78 (with no scrubbing cap 80 thereon). However, in the syringe assembly 12 of FIGS. 15 and 16, rather than the plunger 38, receiving cavity 50, and finger grip 54 of access device 20 being formed as a single integral component, the receiving cavity 50 and finger grip 54 may be fabricated separately from plunger 38 and thereafter connected to the plunger 38. In the illustrated embodiment, plunger 38 includes a female luer connection 118 at the proximal end 46 thereof, while access device 20 (i.e., receiving cavity 50 thereof) includes a corresponding male luer connection 120 thereon that is configured to mate with female luer connection 118 to secure access device 20 to plunger assembly 18 and place the channel 48 of plunger 38 in fluid communication with receiving cavity 50. A collapsible sleeve 122 may be provided that extends through the luer connections 118, 120, with the collapsible sleeve 122 providing a leak-proof fluid path between the channel 48 and the fluid access component 52 (i.e., needle 64) of access device 20. The needle 64 of access device 20 is inserted through the male luer connection 120 to engage collapsible sleeve 122.

While several embodiments of a system configured for line draw flushing and blood collection were described in the foregoing detailed description, those skilled in the art may make modifications and alterations to these embodiments without departing from the scope and spirit of the invention. That is, it is recognized that features of each system of each embodiment may be incorporated into the system of other embodiments. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. A system for line draw flushing and blood collection, the system comprising:
a syringe assembly including:
a pre-filled syringe including a barrel having a distal end and a proximal end and that defines a chamber containing a solution therein, and a fluid connector component at the distal end of the barrel;
a plunger assembly including a plunger having a proximal end and a distal end, and a stopper positioned at the distal end of the plunger that is insertable into the proximal end of the barrel and slidable therein, the plunger including a channel extending along a length thereof between the proximal end and the distal end;
an access device positioned at the proximal end of the plunger, the access device comprising a receiving cavity and a fluid access component, the receiving cavity having an opening therein aligned with the channel, and the fluid access component positioned over the opening and extending into the receiving cavity, the fluid access component in fluid communication with the channel;
a cannula positioned at least partially within the barrel and extending through the stopper, with the cannula configured to place the fluid connector component in fluid communication with the channel; and
a closure assembly secured to the fluid connector component, the closure assembly comprising a seal member configured to seal off a lumen of the fluid connector component to contain the solution within the chamber of the syringe.

2. The system of claim 1, wherein the closure assembly comprises an end cap configured to engage the fluid connector component, wherein the fluid path seal is positioned within end cap, and wherein a proximal end of the end cap comprises a luer fitting configured to mate with an associated luer fitting of the fluid connector component.

3. The system of claim 2, wherein the closure assembly further comprises a scrubbing cap secured to a distal end of the end cap, the scrubbing cap comprising:
a housing secured to the end cap and defining a cavity;
a scrubbing insert positioned within the cavity; and
a seal attached over the cavity to seal the scrubbing insert within the cavity.

4. The system of claim 3, wherein the scrubbing insert comprises a resilient material including an antimicrobial solution or agent absorbed therein configured to disinfect a surface of the access port of the vascular access device.

5. The system of claim 4, wherein the housing comprises a connector portion including a threaded connection configured to engage with the end cap via a twist-type engagement.

6. The system of claim 1, wherein the closure assembly comprises a peel-off web coupled directly to the fluid path seal, to aid in removal of the fluid path seal from the fluid connector component.

7. The system of claim 1, further comprising a container defining a reservoir, the container positionable within the receiving cavity and engageable with the fluid access component to transfer fluid into the reservoir.

8. The system of claim 7, wherein the fluid access component comprises a needle defining a lumen therein, and wherein the container comprises a cover pierceable by the needle to place the reservoir in fluid communication with the channel of the plunger.

9. The system of claim 7, wherein each of the receiving cavity and the proximal end of the plunger includes a luer connection, the luer connection of the receiving cavity engaging the luer connection of the plunger to secure the access device to the plunger assembly, and wherein a collapsible sleeve extending through the luer connections places the channel in fluid communication with the fluid access component.

10. The system of claim 1, wherein the plunger and barrel comprise a locking feature positioned and configured to lock the plunger relative to the barrel when the plunger assembly is in a distally advanced position.

11. The system of claim 1, wherein the plunger and barrel comprise a locking feature positioned to lock the plunger relative to the barrel when the plunger assembly is in a proximally retracted position.

12. The system of claim 1, wherein the cannula is coupled to the barrel and remains stationary during use of the syringe assembly.

13. The system of claim 1, wherein the cannula is coupled to the plunger, such that the cannula is movable responsive to advancing or retracting of the plunger within the barrel.

14. A method of using a system for line draw flushing and blood collection, the method comprising:
providing a syringe assembly including:
a pre-filled syringe including a barrel having a distal end and a proximal end and that defines a chamber containing a solution therein, and a fluid connector component at the distal end of the barrel;
a plunger assembly including a plunger having a proximal end and a distal end, and a stopper positioned at the distal end of the plunger that is insertable into the proximal end of the barrel and slidable therein, the plunger including a channel extending along a length thereof between the proximal end and the distal end;
an access device positioned at the proximal end of the plunger, the access device comprising a receiving cavity and a fluid access component, the receiving cavity having an opening therein aligned with the channel, and the fluid access component positioned over the opening and extending into the receiving cavity, the fluid access component in fluid communication with the channel;
a cannula positioned at least partially within the barrel and extending through the stopper, with the cannula configured to place the fluid connector component in fluid communication with the channel; and
a closure assembly secured to the fluid connector component, the closure assembly comprising a seal member configured to seal off a lumen of the fluid connector component to contain the solution within the chamber of the syringe;
removing the closure assembly from the fluid connector component;
coupling the fluid connector component to an access port of a vascular access device, to place the syringe assembly in fluid connection with the vascular access device;
distally advancing the plunger assembly to flush the vascular access device with the solution;
obtaining a diversion or discard blood volume via operation of the syringe assembly; and
subsequent to obtaining the diversion or discard blood volume, obtaining a sample blood volume via operation of the syringe assembly.

15. The method of claim 14, wherein the closure assembly further comprises:
an end cap configured to engage the fluid connector component, wherein the fluid path seal is positioned within end cap; and
a scrubbing cap secured to a distal end of the end cap, the scrubbing cap comprising:
a housing secured to the end cap and defining a cavity;
a scrubbing insert positioned within the cavity; and
a seal attached over the cavity to seal the scrubbing insert within the cavity;
wherein, prior to removing the closure assembly from the fluid connector component, the method further comprises:
positioning the scrubbing cap over the access port of the vascular access device, such that the scrubbing insert contacts the access port; and
cleaning the access port with the scrubbing insert.

16. The method of claim 14, wherein obtaining the diversion or discard blood volume comprises proximally retracting the plunger assembly within the barrel, to draw the diversion or discard blood volume into the chamber of the barrel.

17. The method of claim 14, further comprising locking the plunger in place relative to the barrel subsequent to proximally retracting the plunger assembly to draw the diversion or discard blood volume into the chamber of the barrel.

18. The method of claim 14, wherein obtaining the diversion or discard blood volume comprises:
positioning a container within the receiving cavity, the container defining an evacuated reservoir therein;
engaging the container with the fluid access component to draw the diversion or discard blood volume into the evacuated reservoir; and
removing the container from the receiving cavity.

19. The method of claim 14, wherein obtaining the sample blood volume comprises:
positioning a container within the receiving cavity, the container defining an evacuated reservoir therein;
engaging the container with the fluid access component to draw the sample blood volume into the evacuated reservoir.

20. The method of claim 14, wherein the fluid access component comprises a needle having a lumen therein, and where engaging the container with the fluid access component comprises piercing a cover of the container with the needle, to position the needle partially within the reservoir.
